(19) United States
(12) Patent Application Publication
(10) Pub. No.: US 2019/0156947 A1
(43) Pub. Date: May 23, 2019

(54) AUTOMATED INFORMATION COLLECTION AND EVALUATION OF CLINICAL DATA

(71) Applicant: Vital Images, Inc., Minnetonka, MN (US)

(72) Inventors: Eve Megumi Nakamura, Bloomington, MN (US); Osama Masoud, Woodbury, MN (US); Oliver Schreck, Chaska, MN (US)

(21) Appl. No.: 15/821,182

(22) Filed: Nov. 22, 2017

(51) Int. Cl. G16H 50/20; G16H 30/40

(52) U.S. Cl. CPC G16H 50/20; G16H 30/40

(57) ABSTRACT

Techniques and system configurations to enable the automated collection and evaluation of clinical data within an automated insights information system are disclosed herein. In an example, the information system is adapted to continuously monitor clinical systems for new patient data, process patient data into a standardized and structured format, selectively run algorithms to classify and characterize data, and stores the results of algorithms (such as findings, predictions, and recommendations) that can be used as input to other algorithms, or sent to clinical systems and presented to end users. In a specific example, a method performed in a computing system may include: requesting and obtaining a first and second set of clinical data, analyzing the first and second set of clinical data with respective algorithms, identifying a clinical finding, and generating output from the computing system based on the identified clinical finding.

### TECHNICAL FIELD

Embodiments pertain to data processing techniques and configurations used with information networks and informatics systems. Further embodiments relate to the use and execution of specialized information analysis algorithms and artificial intelligence processes used in medical diagnostic and evaluative settings.

### BACKGROUND

As healthcare processes have become increasingly digitized, large volumes of clinical data is now generated on human patients at nearly every medical facility for many types of healthcare interactions. However, as the volume of data has increased, the complexity of retrieving, interpreting, and drawing useful conclusions from the data points in such clinical data has also increased. This challenge is caused, in part, from the variability of the amount and type of clinical context available from data for a given patient. This variability may ultimately result in uncertainty for the clinical treatment and diagnosis of the given patent.

The digitization of healthcare has provided many opportunities for computer-based systems, including computer-assisted detection (CAD) and clinical decision support systems, that can help clinicians work more efficiently. Advances in machine learning, artificial intelligence, and computer hardware have also enabled the development of efficient algorithms and models that can efficiently process and learn patterns from massive quantities of unstructured data. However, the accuracy of such algorithms and models is often limited based on correct deployment and usage, which is likewise complicated by the variability of the amount and type of clinical context available from data for a given patient. Limited approaches and workflows have been utilized to customize the base of medical information for a given patient and define types of actions and outputs that can be taken when certain conditions are detected. However, such approaches are often implemented with manual programming and strict protocols, and have limited applicability to a specific type of clinical use case.

### BRIEF DESCRIPTION OF THE DRAWINGS

FIG. 1 illustrates a diagram of a system configuration for collecting, processing, and outputting medical information according to an example described herein.

FIGS. 2A and 2B illustrate data flows for generating and outputting medical information data from among various types of patient data according to an example described herein.

FIG. 3 illustrates a diagram of a cascaded approach for collecting and processing medical information data with multiple algorithms according to an example described herein.

FIG. 4 illustrates a further diagram of a cascaded approach for processing and outputting medical information data according to an example described herein.

FIG. 5 illustrates a flowchart of sequential data processing operations performed for obtaining and analyzing data in a clinical data analysis system according to an example described herein.

FIG. 6 illustrates a flowchart of a specific method for information processing in a computing system, such as performed by an automated insight information system according to an example described herein.

FIG. 7 illustrates a block diagram of respective computer systems used for information processing, for a use case of automated insight information processing according to an example described herein.

FIG. 8 illustrates an example of a machine configured to perform computing or electronic processing operations according to an example described herein.

### DETAILED DESCRIPTION

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments.

The present disclosure illustrates various techniques and configurations within an information system to enable the collection, analysis, and monitoring of clinical or other medical-related data from disparate data sources. The various techniques and configurations, as applied to clinical (e.g., medical or medical condition) data, include aspects of an automated system that continuously monitors clinical systems for new patient data, processes patient data into a standardized, structured format, selectively runs algorithms (e.g., artificial intelligence models or rule-based processes) to classify and characterize data, and stores the results of the executed algorithms. The results of these algorithms may include findings, predictions, and recommendations, which can be used as input to other algorithms, stored within the information system, or sent to clinical systems and presented to users. As more patient data and algorithm results become available, the information system continues to refine its findings, predictions, and recommendations, thereby increasing the extent of clinical decision support provided.

The following operations and system configurations may be used to address a number of technical problems within computer and information processing fields, and related information classification problems within clinical applications of information technology. In the context of the following examples, some of the technical problems addressed include: prioritized and efficient processing of a large volume and large number of sources of clinical data; suitable processing prioritization and data-driven workloads based on relevancy and timeliness conditions; accuracy of such technological and clinical workflows and the accompanying computer data operations (e.g., to reduce errors, reduce the amount of data being processed, and reduced the amount of time needed to process); variation in clinical interpretation and diagnostic findings, which result in variance, erroneous data, or unpredictable results; and the accessibility of resources (both computer resources and medical resources) which often leads to shortages and inefficiencies.

As discussed in the following examples, specific reference is made to a number of medical diagnostic, interpretation, and treatment settings. However, it will be understood that the technical problems addressed with the present disclosure are not limited to medical or business improvements; rather, the disclosed processing techniques address the functioning of underlying medical information systems and data processing networks that are essential to modern medical practices. Thus, the application of the present techniques to data analysis in other scientific and academic disciplines and technical fields will be apparent.

In the context of medical information, the present techniques may generate automated insights from system operations that automatically and continuously classify, characterize, and integrate patient and clinical data to produce and refine findings, predictions, and recommendations (collectively, such findings, predictions, and recommendations are referred to herein as “insights”). The insights that are produced may continuously integrate new patient data and algorithm results, as respective findings, interpretations, predictions, and recommendations are integrated accordingly. As a result, when new patient or clinical data or algorithm results are introduced into the system (or otherwise become available), the system may evaluates rule and enforces prerequisites, such as to only run applicable algorithms. In certain examples, these applicable algorithms may be executed only once, and on an as-needed basis. This results in the conservation of computing system resources, while also supporting scalability.

These and other data processing approaches discussed herein provide a number of benefits that extend beyond medical informatics into a variety of technical fields. With the use of the present techniques, computing processing of medical information data sets may be automated and improved significantly. Such computing may result in improved automation of previously manual activities, causing a reduction in errors and inaccurate activities, and improved efficiency of software operations and associated processing and networking hardware resources. Further, the techniques provide improvements over conventional approaches for collecting and analyzing related medical information data content, allowing improved computational results and improved graphical outputs in reduced time.

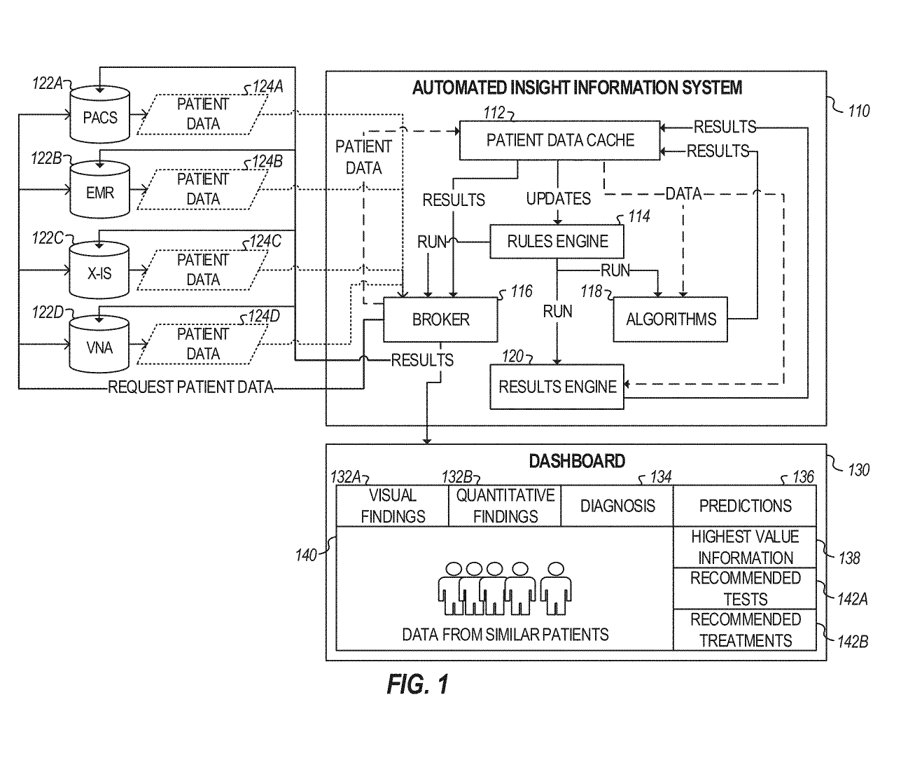

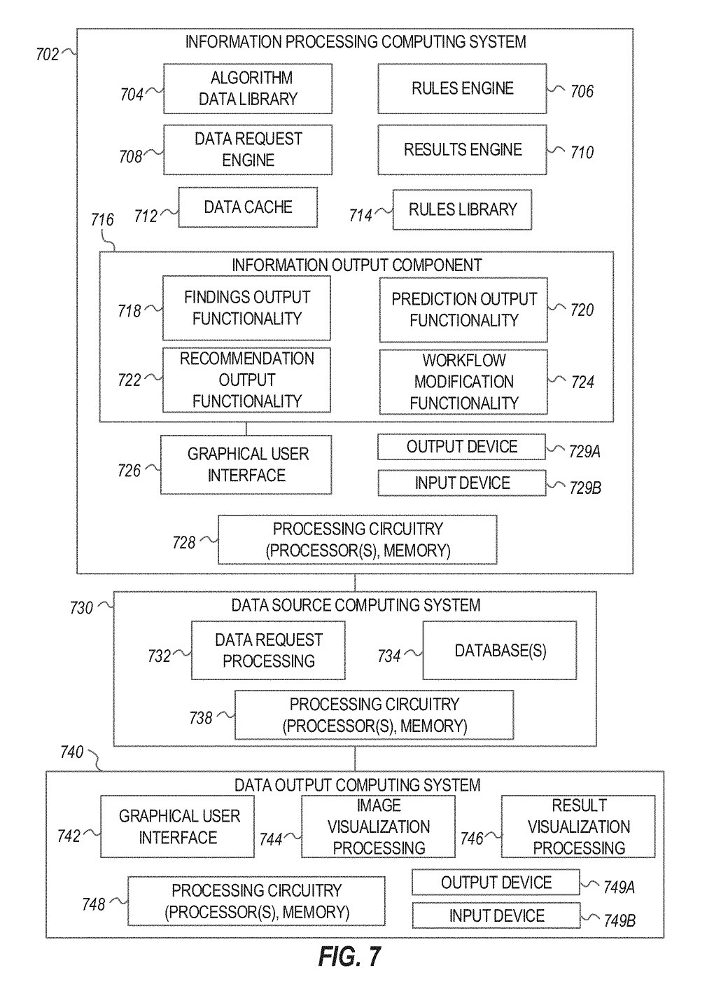

FIG. 1 illustrates an overview diagram of an example system configuration for collecting, processing, and outputting medical information, using the present information system approaches. Specifically, FIG. 1 illustrates features of an automated insight information system 110, which includes a number of sub-components (patient data cache 112, rules engine 114, broker 116, algorithms 118, results engine 120). It will be understood that the system configuration of FIG. 1 is arranged into high-level components for purposes of illustration, and individual features of the depicted components may be integrated or separated among various machines, systems, and devices. An example multi-system configuration is discussed, for instance, with reference to FIG. 7, below. However, many of the processes and functions depicted in the following example may be simulated or replicated by like functions within consolidated or separated device subsystems.

As shown in FIG. 1, the automated insight information system 110 operates to coordinate data-driven operations within and from outside the system 110, as the system 110 requests and receives data inputs from clinical systems (e.g., clinical data sources 122). Further, the system 110 processes and evaluates the obtained data through use of the patient data cache 112, the rules engine 114, the broker 116, the algorithms 118, and the results engine 120, and the system 110 generates and provides an output to via a dashboard system 130. The data requests and inputs into the system 110 may occur in an iterative fashion, as discussed below, as the operation of the respective algorithms leads to data results and changes, that in turn, leads to more input data that is analyzed with further data results and changes. Accordingly, although not directly depicted, multiple and sequential or repeating requests, operations, and responses, may be occurring for a collection of patient data.

A processing workflow in the system 110 may be initiated by a request for analysis of a particular medical data set, using an initial set of patient data. As discussed below, the broker 116 operates to request and receive patient data 124 from one or more clinical data sources 122, and the patient data 124 is then hosted in a patient data cache 112 for further processing within the system 110 with use of one or more algorithms 118. The execution of the algorithms 118 is coordinated by the rules engine 114, which schedules the execution of the algorithms 118 and coordinates the provision of the patient data from the patient data cache 112 to the algorithm. In turn, the respective results of the algorithms 118 are stored in the patient data cache 112. The results of the algorithms 118 may then be further processed and propagated via a results engine 120.

In an example, the patient data 124 may include, but is not limited to, the following forms of clinical data: images (e.g., DICOM studies, key images, secondary image captures); patient demographics (e.g., age, gender, ethnicity); medical history; medical status(es) (e.g., a listing of prescriptions, medications, devices, implanted devices, etc.); radiology reports; laboratory test results; pathology reports; family history information; genomics data; proteomics data, and the like. The patient data 124 may be provided in a variety of data formats including as binary image data, codes, numerical values, or alphanumeric text. The patient data 124 is not limited to data generated by a medical professional or medical facility in a clinical setting; rather, some of the data may be provided by users directly, by third parties, or as a result of information system processing.

As depicted, clinical systems that may provide the patient data 124 include, but are not limited to: a Picture Archiving and Communication System (PACS), an Electronic Medical Record (EMR) or Electronic Health Record (EHR) system, an information system (e.g., Health Information System (HIS), Radiology information system (RIS)), or a Vendor Neutral Archive (VNA). In a specific example, the patient data may include two- or three-dimensional image data of a human subject, such as image data acquired from an imaging modality (e.g., an x-ray machine, computed tomography (CT) scanner, magnetic resonance imaging (MRI) machine, and the like), also referred to as radiology imaging data.

In a specific example, the patient data 124A may be directly or indirectly provided from a specialized medical imaging system such as a PACS data store 122A. In another specific example, the patient data 124B may be provided from an EMR data store 122B. Also in a specific example, the patient data 124C may be provided from a specialized information system, such as a RIS, HIS, or other similar health-related information system (designated “X-IS”) data store 122C. Also in a specific example, the patient data 124D may be provided from another image data retrieval, caching, or storage system such as a VNA data store 122D. The format of the patient data 124 (e.g., patient data 124A) may be in a proprietary or industry standard format, such as in a Digital Imaging and Communications in Medicine (DICOM) format for medical imaging data. Although some of the following examples refer to the processing of radiology imaging data, the following techniques may be used with other types and format of the patient data 124.

The information system 110 operates to use the various components 112, 114, 116, 118, 120 to request and collect the patient data 124, store a copy of the patient data 124 in the patient data cache 112, analyze the patient data with particular algorithms 118, persist the results of the analysis from the particular algorithms 118 in the patient data cache 112, and provide the results via a dashboard 130. In an example, the information system 110 operates these components as follows:

Algorithms. The algorithms 118 are self-contained, standalone components (e.g., modules, plug-ins, software libraries) that may be executed within the system 110 for a particular information evaluation, classification, or evaluative purpose. In an example, the algorithms 118 are selected and run by the rules engine 114 based on a successful evaluation of algorithm rules (e.g., prerequisites, or conditions) relative to the current data available in the patient data cache 112. The algorithms 118 take data and results from the patient data cache 112 as input, and the algorithms 118 provide output results that are stored as results in the patient data cache 112. Any number of algorithms 118 may be installed or enabled for use by the system 110, and the rules engine 114 operates to ensure that algorithms are executed when appropriate.

In an example, each of the algorithms 118 performs one classification or characterization task. Each algorithm is associated with a set of rules (prerequisites), including required inputs, and is not run unless all required inputs for the algorithm are currently in the patient data cache 112. The algorithms 118 also may take optional inputs to increase confidence in the produced conclusions. As an example, an algorithm for aortic aneurysm analysis may be associated with the following rules: (1) study modality must be CT; (2) abdominal region must be present; (3) contrast must be present within the aorta. The following inputs are not required for this algorithm, but may be used as additional data inputs: (a) age; (b) gender; (c) smoker; (d) height/weight; (e) medical conditions (e.g. hypertension, high cholesterol, diabetes); (f) patient complaints (e.g. abdominal pain); (g) family history; (h) prior studies.

Result outputs that are produced by the algorithms 118, for a respective algorithm execution, may include, but are not limited, to: Boolean flags (yes/no, relative to some condition); images and image data (e.g., key images, image series, secondary captures, which are selected, modified, or produced); qualitative or quantitative measurements (e.g., length, angle, diameter, volume, surface area, sphericity, density, heterogeneity of density); text (e.g., a structured summarization of free text, or annotations of medical data); and other forms of findings, predictions, and recommendations, relative to a medical condition, a diagnostic state, or the like.

The algorithm results may be stored in the patient data cache 112, and may or may not constitute clinical (e.g., diagnostic) findings. Examples of algorithms that may not produce clinical findings may include landmark detection or contrast detection algorithms. Examples of algorithms that may potentially produce clinical findings include: an automatic aortic aneurysm algorithm, a Brain CT Hematoma algorithm; a coronary artery calcium scoring algorithm; or, a colon computer-aided detection algorithm.

Broker.

The broker 116 communicates with external data sources and systems (e.g., clinical data sources 122A-122D), and obtains and coordinates data among input and output locations. In an example, the broker 116 operates to: monitor clinical systems and observe when new patient data becomes available. Examples of new patient data becoming available include: a new imaging study arrives at the PACS data store 122A, results from laboratory tests appear in the EMR data store 122B, a new radiology report appears in the RIS data store 122C, a progress report appears in the EMR 122B, and so forth.

The broker 116 may further fetch or capture patient data from clinical systems, process the patient data (e.g., by anonymizing, extracting relevant features, converting into a structured format), and update the patient data cache 112. In an example, the broker 116 fetches patient data when new patient data becomes available, or the rules engine 114 requests patient data that is not already in the patient data cache 112. For instance, the rules engine 114 may request data from “similar” patients; that is, patients from the same or similar demographic groups, patients with the same or similar medical conditions or medical histories, patients who have undergone the same or similar procedures or treatments, and so forth.

In a further example, the broker 116 may also provide results (findings, predictions, recommendations) to clinical systems (including the clinical data sources 122) or an output location (such as the dashboard 130) for presentation to users.

Patient Data Cache.

The patient data cache 112 stores patient data, as it is received and processed within the system 110. In an example, this data is modified patient data that is requested and anonymized by the broker 116. In a further example, the anonymized, structured patient data from the broker 116, results from algorithms 118, and results from the results engine 120, are each stored in the patient data cache 112.

Rules Engine.

The rules engine 114 manages the selection, invocation, and operation of the various algorithms 118. In an example, the rules engine 114 includes logic and configurable rules to: register algorithms that have been installed on the system; track and organize rules (e.g., conditions and prerequisites) for running each algorithm; monitor the patient data cache 112 for updates; evaluate rules and select appropriate algorithms to run when the patient data cache 112 is updated; schedule updates (e.g., algorithm changes) that are sent to or retrieved from an external system (e.g., a distributed learning system).

In an example, the rules engine 114 continuously monitors the patient data cache 112 for updates, including new or updated patient data, algorithm results, findings, predictions, recommendations, and the like. When the patient data cache 112 is updated, the rules engine 114 may run one or more of the algorithms 118, request that the broker 116 to fetch more patient data, or request the results engine 120 produce refined, updated, or supplemental findings, predictions, and recommendations. These updates may subsequently result in updates to the patient data cache 112.

In an example, each algorithm is associated with a set of rules (prerequisites). Examples of rules (prerequisites) include the following: modality of an imaging study must be CT; results from white blood cell count laboratory tests performed within the last 7 days must be available; prescription for statins must exist; patient age must be at least 65 years old; results from a landmark detection algorithm must indicate the presence of landmarks in the abdominal region; and the patient must have an endovascular stent.

Without prerequisites, the same algorithms may be run multiple times given the same patient data at the same point in time. In the system 110, each algorithm is registered with the rules engine 114, along with the algorithm prerequisites. Given the same patient data at a particular point in time, each particular algorithm can be run just once. The results of each particular algorithm are cached and used to determine which further algorithms are appropriate to run. Checking and enforcing prerequisites ensures that algorithms are not run unless necessary, which conserves system resources and supports scalability.

Results Engine.

The results engine 120 continuously integrates new patient data and results (including new results that the rules engine 114 produces) from the patient data cache 112 to produce findings, predictions, and recommendations, with these findings, predictions, and recommendations then being stored or updated in the patient data cache 112. The extent of clinical decision support provided by the results engine 120 depends on what relevant patient data and results are currently available, and the capabilities of the algorithms 118 that are identified and selected for execution. As more patient data and results are stored in the patient data cache 112, the results engine 120 further refines the available results; further, as more patient data and results are stored, additional preconditions for execution of additional algorithms 118 will be satisfied with the rules engine 114; leading to increasing accuracy and coverage of information evaluation operations of generated results.

As an example, if all image analysis algorithms that are registered with the system 110 have specific requirements with respect to anatomical landmarks and the presence or absence of contrast, the rules engine 114 may automatically run a landmark detection algorithm and a contrast detection algorithm when any imaging study becomes available, cache the results of each algorithm, and based on the results, select appropriate algorithms 118 to run. Updates to the patient data cache 112 may further trigger reevaluation of the rules for automatically running algorithms. Based on the data that is hosted in the patient data cache 112, the rules engine 114 may perform any of the following: select and run one or more of the algorithms 118; request that the broker 116 obtain additional patient data for the same patient or for specific patients or types of patients (e.g. similar patients); or request that the results engine 120 refine its findings, predictions, and recommendations.

Dashboard.

As shown, the system 110 may be operatively coupled to a dashboard 130 or other output system. The dashboard 130 may operate to display, visualize, or otherwise output insights produced from the system 110, with one or more of the following: visual findings 132A (e.g. key images of detected pathologies); quantitative findings 132B (e.g. measurements and locations of detected pathologies); diagnosis information 134 (e.g., disease classification, and staging); predictions 136 (e.g., prognosis, estimated disease recurrence rate, patient survival rates); highest value information 138 (e.g., what actions would contribute the most to increasing the quality of the predictions); recommended tests 142A (e.g., a list of additional medical tests or diagnostic procedures that may produce additional data); and recommended treatments 142B (e.g., a list of relevant treatment information, based on the diagnosis information 134, and the predictions 136). In a further example, the dashboard 130 may provide a comparison of the analyzed data to patient data for “similar” patients 140.

In an example, the features of the dashboard 130 may be integrated within an image visualization system (not shown), allowing the visualization of medical imaging data in addition to aspects of the visual findings 132A. For instance, aspects of data visualization in the image visualization system may be used to display and change (e.g., augment, highlight, change, modify, remove, segment, etc.) features of medical images obtained from the clinical data sources 122. Further, the data visualization may be used to output a representation of the medical images, the visual findings 132A, and the quantitative findings 132B, via a clinical graphical user interface output (also not shown). For example, such a graphical user interface output may be provided via a display device (e.g., monitor, screen, etc.) connected to the image visualization system.

Manually querying conventional clinical systems to retrieve many different data points can be tedious and time-consuming. This involves searching for and reading through large quantities of free text in order to extract relevant information and introduces room for inconsistencies (e.g., especially as one clinician has more, or different, information than another clinician for the same patient). To address these issues, the dashboard 130 aggregates, summarizes, and displays relevant patient data, visual and quantitative findings, interpretations of findings, clinical best practices and guidelines (e.g., medical group guidelines), and recommendations in a structured form and in a centralized, easily accessible location so that all clinicians have the same information available to them.

In further examples, the operations from the information system 110 may be utilized to collect and persist feedback regarding the operation of the algorithms 118, including feedback from the operation of machine learning models at the local system 110 and from other connected systems. For instance, the system 110 may integrate with a distributed set of deployed algorithms to send updates to and receive algorithm updates (e.g., machine learning model weights) from an external system. Thus, the use of the system 110 may operate in the context of a machine learning model that is deployed among a plurality of information systems and clinical deployments, to allow training top occur in a real-world, interactive fashion. The algorithms employing such models may be updated continuously as training data from other sites becomes deployed to the information system 110.

In further examples, the operations from the information system 110 may be utilized to affect a medical diagnostic or interpretation workflow. In this context, such a workflow may refer to a series of operations performed with some combination of manual (human-specified) and automated activity, to perform a task for a medical-based purpose. One example of a workflow in the medical imaging field of use is a radiology read workflow, where a series of diagnostic images (e.g., produced by an imaging modality) are evaluated by a reviewing medical professional (e.g., a radiologist) to produce a diagnosis directly from the medical images. Thus, the information techniques discussed herein may be integrated to allow algorithm-derived information to be presented (e.g., output, displayed, annotated, recorded) within the interactive activities of the radiology read workflow.

Another example of a workflow in the medical imaging field of use is an image review workflow, where captured medical images may be manipulated, viewed, or evaluated by a clinician (e.g., a specialist, doctor, or other trained professional), such as to perform segmentation, quantification, or prediction of anatomical features of a medical image to confirm a diagnosis or observe the state of a particular medical condition. Other examples of workflows may involve other types of actions, feedback, and control that occur from interaction with a human user or users in a software application or accompanying computer system, a medical condition, diagnostic, or other evaluative settings.

The information determined from the algorithms 118 and the accompanying findings, diagnosis, prediction, and recommendation information, may be integrated into these and many other clinical settings. Further, the automated or computer-assisted workflow operations for automated medical insight evaluation may be applicable in both medically diagnostic and non-diagnostic (e.g., informational, educational, demonstrative, testing, etc.) settings.

As will be apparent, the operation and deployment of the information system 110 may address a number of limitations and challenges within the technical field of medical informatics data processing. Some of the technical limitations that are addressed through the present techniques and configuration include:

Large Volumes of Clinical Data.

The volume of clinical data generated by electronic medical systems for each patient continues to increase over time, causing the need to efficiently extract relevant features and produce interpretations from many different data points that are scattered across many different clinical systems. This has resulted in more clinical data that is generated than can be interpreted by humans. The present techniques in the system 110 apply intelligence to identify which data should be analyzed, request the data on demand, operate a data-appropriate set of algorithms, and efficiently track results based on the amount of available data and available algorithms.

Increased Workloads.

Increased utilization of cross-sectional imaging has resulted in increases in the number of images acquired per imaging study and the frequency of discovery of findings that are unrelated to the reasons for which the study was performed, which contribute to increased workloads for radiologists and other clinicians. Further, there is often resource shortage for medical professionals to analyze all data fields and data records. As the workloads of radiologists and specialists continue to increase, effective management and prioritization of worklists and worklist activities becomes even more important. In a similar fashion, as more algorithms become available, limited computer resources and time may prevent brute-force evaluation of all available algorithms against incomplete data sets.

Diagnostic Errors.

Diagnostic errors, including missed findings, are common and may have serious consequences for patients and clinicians. Many factors contribute to diagnostic errors, including cognitive biases, visual and mental fatigue, and errors and inefficiencies in clinical systems and processes. Conventional approaches do not prioritize the application of algorithms and data results in an attempt to improve accuracy.

Variability of Interpretive Accuracy.

Studies have shown that there can be significant variability among radiologists with respect to interpretive accuracy. Many types of detection tasks are difficult and may therefore benefit from having more than one reader. The techniques used within the system 110 may provide a consistent approach for data evaluation and use of algorithms.

Clinical Context.

Radiologists and other clinicians may not be provided with a full clinical context when interpreting medical images, which reduces the efficiency and interpretive accuracy of such interpretations. Further, radiologists are oftentimes responsible for not only documenting significant findings in reports but also following up on them, which can be difficult and time-consuming. The intelligent deployment of the algorithms 118 within the system 110 may significantly assist within clinical decision support and improvement of accuracy in these and other clinical contexts.

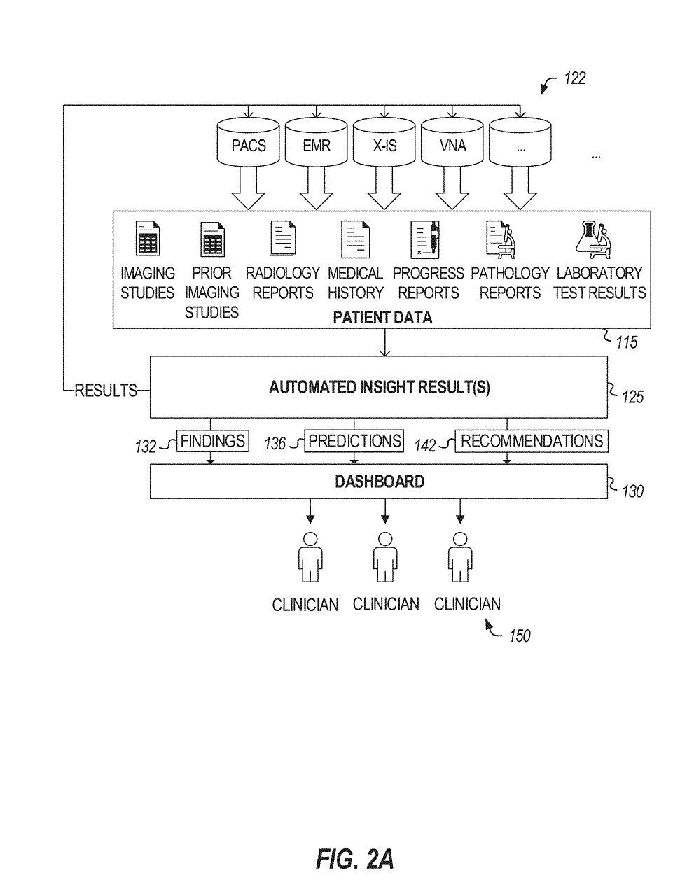

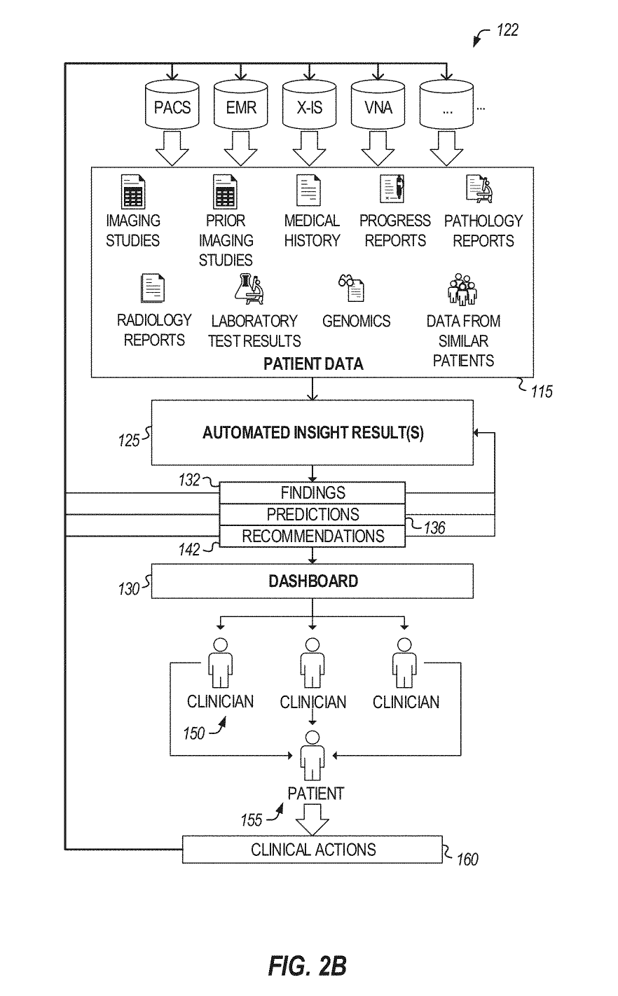

FIGS. 2A and 2B illustrate example data flows for generating and outputting medical information data from among various types of patient data according to an example described herein. As shown, FIG. 2A illustrates a simplified process for the analysis of respective types of patient data 115, such that patient data is obtained from the respective data sources 122 and used to produce one or more generated automated insight results 125. From these generated insight results 125, various result data may be stored or updated in the data sources 122, or provided in the form of findings 132, predictions 136, or recommendations 142 to the dashboard 130 or other graphical user interface. In turn, this dashboard 130 may output information to respective clinicians 150.

In practice, the clinicians 150 often must quickly find connections between many data points stored in disparate clinical systems to understand the clinical context when providing care to patients. Retrieval, extraction of relevant features, and interpretation of this information can be tedious, difficult, and time-consuming. Variability in the extent of clinical context available may result in variability of quality of interpretation, which is undesirable and detrimental to patient outcomes. Furthermore, different clinicians may consider different subsets of clinical data to be relevant. The generation and use of the automated insight results 125 enables the collection of patient and clinical data as possible from as many clinical systems as possible to maximize clinician efficiency, as the system 110 continuously refines the extent of clinical decision support that it provides by monitoring for and integrating new patient data and results from algorithms.

FIG. 2B shows a similar process, but with the illustration of how actions performed by the clinicians 150, relative to treatment of a patient 155, result in various clinical actions 160 (e.g., the administration of treatment, additional tests, etc.). These clinical actions in turn will result in additional data being captured and stored with the data sources 122, and additional data for subsequent generation of the automated insight results 125.

Accordingly, the automated insight results 125 may result in automated aspects of workflow efficiency and consistency. In an example, the automated insight results 125 maximizes workflow efficiency and consistency by automatically and continuously classifying, quantifying, and characterizing findings and pathological conditions as new patient data becomes available, which facilitates prioritization aggregating, summarizing, and interpreting patient data, algorithm results, and clinical best practices and guidelines and preparing this information for presentation in a centralized place (e.g., the dashboard 130) for ease of comprehension by clinicians.

Also in a further example, the automated insight results 125 may support clinical decisions and determinations in the treatment of the patient 155 by the clinicians. The automated insight results 125 provide clinical decision support by continuously making and refining predictions and recommendations based on changes in patient data and results from algorithms. As more patient data and algorithm results become available, the level of clinical decision support and data accuracy increases.

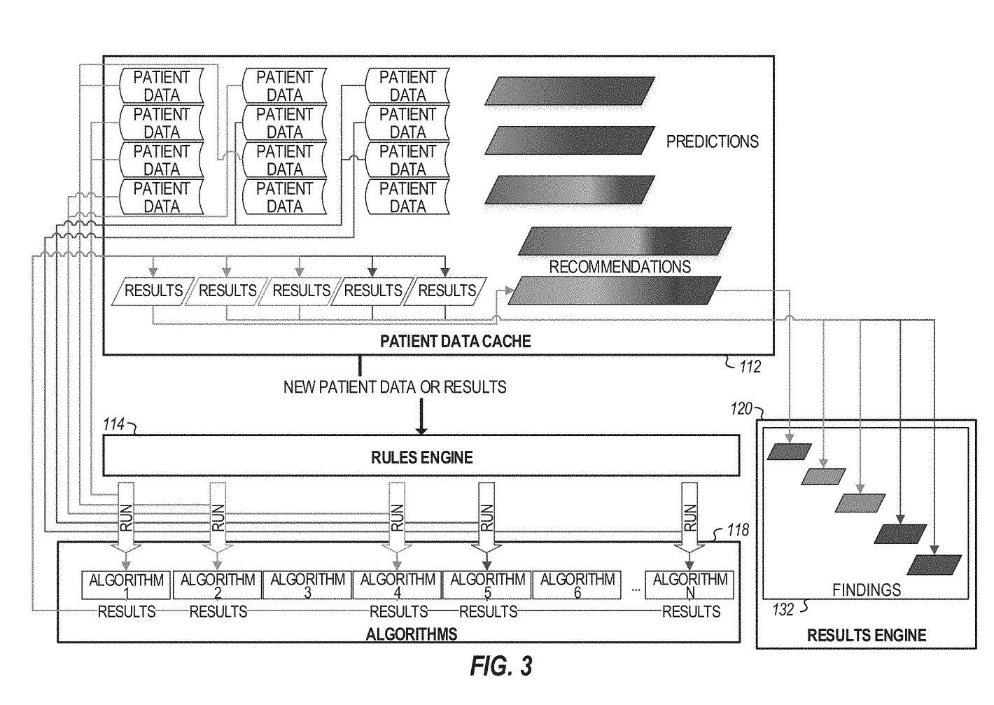

FIG. 3 illustrates a diagram of a cascaded approach for collecting and processing medical information data with multiple algorithms according to an example described herein. FIG. 3 specifically illustrates the collection of a series of patient data results stored within the patient data cache 112, after the patient data results are obtained from one or more patient data sources (e.g., the data sources 122). As new patient data or results are made available in the patient data cache 112, the rules engine 114 operates to identify, select, and operate various algorithms 118. The results produced from the algorithms 118 is then stored in the patient data cache in respective sets of results.

A number of patient data results may be produced from the operation of multiple of the algorithms 118. For instance, as shown in FIG. 3, some (but not all) of the N algorithms are used to produce five sets of results, which are communicated back to the patient data cache 112. These results may be in the form of predictions, recommendations, or findings, as related to a particular medical condition, anatomical area, diagnostic or evaluative condition, or similar data point.

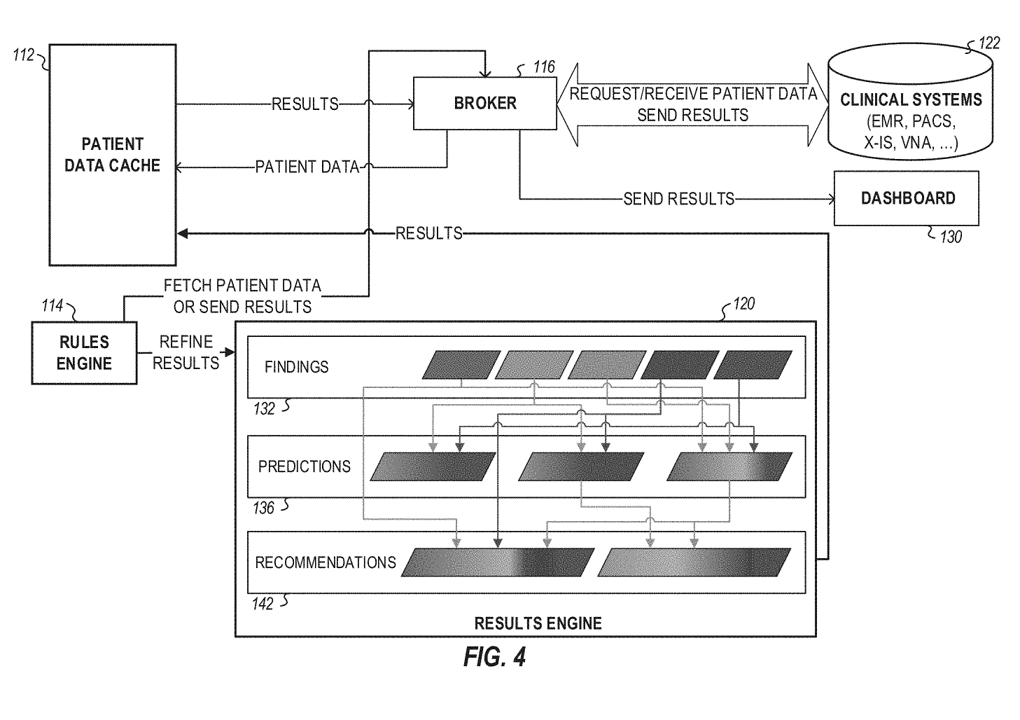

FIG. 3 also illustrates the output of the various results from the patient data cache 112 to the results engine 120 (with the five sets of results being communicated to the results engine 120). A further depiction of the use and processing of the results with the results engine 120 is illustrated in FIG. 4, and discussed further below.

Returning to FIG. 3, the information system 110 continuously refines findings, predictions, and recommendations based on new patient and clinical data and algorithm results produced within the system. This refinement occurs through the generation of iterated sets of results, controlled via the rules engine 114. As a result, the information system 110 continuously integrates new patient data and algorithm results and refines its findings, interpretations, predictions, and recommendations accordingly.

In an example, the rules engine 114 selects and runs algorithms based on the successful evaluation of algorithm rules (e.g., requirements, prerequisites, conditions) against the current contents of the patient data cache 112 or other data inputs. To conserve system resources and support scalability, a respective algorithm from the algorithms 118 is executed only when all rules are satisfied. Thus, as prerequisites are enforced, the applicable algorithms may be executed only once for each new unit of patient data or algorithm result (and need not be re-executed as new data is obtained).

The algorithms 118 may be interchanged or customized to adapt to the detection of particular medical conditions. The algorithms 118 can learn and improve continuously, including based on use of the algorithms at other medical facilities, locations, and institutions. In an example, the algorithms 118 can be automatically run as soon as new patient data or results from other algorithms become available, even before clinicians interpret the new data (e.g., in a radiology workflow). Further, the algorithms 118 can process and integrate large quantities of heterogeneous data from disparate clinical systems.

In a further example, each of the algorithms 118 deployed in the system 110 may be associated with built-in rules to identify new patient data that is appropriate for the pathological condition being classified, so that the system 110 starts a respective algorithm as soon as applicable patient data is available on the system 110. As previously discussed, the rules engine 114 is adapted to automatically execute only appropriate algorithms, thus conserving system resources and reducing false alarms. Also in a further example, various configuration settings may be associated with each of the algorithms 118 such that users can easily configure the algorithms 118 according to clinical use cases, and to customize effects according to specificity and sensitivity.

In an example, the algorithms 118 are provided in the form of defined classifiers that are automatically invoked. For example, each of the classifiers (with one or more classifiers associated with a particular algorithm) may be created to focus on a specific abnormality or diagnostic condition. For instance, each classifier may be responsible for identifying and characterizing one, and only one, type of abnormality. However, there may be many classifiers that are applicable to data produced from a particular modality and from a body region (and thus, multiple classifiers that are invoked by a particular algorithm and data set).

In a further example, each classifier may be trained to characterize a specific abnormality with high specificity and customized sensitivity. For instance, classifiers may be neural networks or other machine learning or defined rule-based algorithms. The algorithm may include high specificity to avoid false alarms; the sensitivity may be set at a sufficiently high level to be useful. In further examples, the classifiers may be configured by users to operate with higher (or, lower) sensitivity.

When new patient data becomes available on the system, the rules engine 114 may be adapted to automatically run all applicable classifiers. This is illustrated by the approach in FIG. 3, which collects a large series of patient data sets, for analysis by multiple of N algorithms (with each of N algorithms including one or more classifiers). As discussed above, the respective patent data sets may encompass data such as imaging studies, EMR data, laboratory test results, pathology reports, genomics, and the like. Every classifier has a set of rules (modality, body region, contrast/non-contrast, and the like) that must be satisfied for the algorithm to be automatically run on a particular piece of patient data. More than one classifier may be run on a piece of the patient data, or no classifier may be run.

As specific example, a coronary calcium classifier, emphysema classifier, and lung nodule classifier may all be executed on a non-contrast lung screening (chest) CT study, while a thoracic aortic aneurysm classifier may have a rule that would require images with contrast enhancement. As another specific example, a knee MR study may have no applicable classifiers, in which case no classifiers will be run. The classifiers that are not applicable to newly available patient data are prevented from running, thus reducing wasteful use of system resources as well as the potential for false alarms.

The output of the algorithm, in the form of findings obtained by the results engine 120, may provide a variety of graphical, textual, or procedural outputs. For instance, the output may provide a graphical indication if any classifier produces a prediction of interest with high probability for a particular condition; these and other findings may be indicated to the user in different ways—alerts and notifications, key images, recommendations, and the like.

FIG. 4 illustrates a further diagram of a cascaded approach for processing and outputting medical information data according to an example described herein.

The diagram of FIG. 4 continues based on the results produced by the algorithms and rules engine in FIG. 3, with the respective results (five sets of results, indicated as findings 132) being provided to the results engine 120. These findings 132 are then further corresponded to various types of predictions 136 and recommendations 142.

The various predictions 136 and recommendations 142 provided by the rules engine may be produced from various permutations of the findings 132. For instance, a first test applied with a first algorithm may produce a first finding, whereas a second test applied with a second algorithm may produce a second, different finding; each finding may be based on one or more of outputs from an algorithm; each prediction may be based on one or more of the findings; each recommendation may be based on one or more of the findings 132 (and, in some cases, one or more of the predictions 136).

The information system 110 can provide many types of results that are useful in clinical workflows and to enhance clinical decision support. In this fashion, the information system 110 may automatically detect, classify, and characterize pathological conditions based on one or more sources of patient data, while predicting the patient outcome and making recommendations suitable for clinical application.

Clinical Decision Support. In a further example, the information system 110 may provide clinical decision support in different scenarios where information is incomplete or unclear. This may be extended with various forms of aggregating, summarizing, and analyzing patient data to maximize information content, minimize reading time, and provide additional insights.

As broad examples of use cases, the techniques performed by the information system 110 may be used to: warn a clinician who is ordering an imaging study that the patient has a contrast allergy and recommend alternative procedures if possible; fetch and display relevant clinical guidelines for the management of a kidney cyst incidentally discovered during an abdominal/pelvis CT study; recommend treatments, additional tests, and a follow-up screening plan for a patient with a 6 mm solid lung nodule; automatically detect brain hemorrhage in a patient who has just undergone a non-contrast head CT study following a car accident and recommend further imaging procedures and clinical care; remind a clinician that a follow-up recommendation was made for a patient but no action has been taken (i.e. no appointment was scheduled, no imaging study was ordered, etc); warn an ordering physician of differences between the clinical indication and the priority of a procedure being ordered (for example, a routine outpatient procedure ordered stat, or a portable chest x-ray exam for endotracheal tube placement ordered non-stat) and display clinical indications for and expected turnaround times for the same procedure when ordered at each priority (stat, today, routine, and so forth).

Thus, the operations of the information system 110 may result in identifying actionable findings and reminding clinicians to follow up on them as appropriate-automatically finding discrepancies with respect to significant findings between diagnostic reports and studies.

The information system 110 may also detect discrepancies between findings reported by humans and findings reported by algorithms. For example, in a conventional human-driven workflow, an imaging study is ordered and performed, a radiologist interprets the study and writes a report, and the report goes to the RIS or other clinical system. There are multiple places where automated insight results may be utilized (noted the following steps (2) and/or (5)): (1) An imaging study is ordered and performed. (2) The information system retrieves the imaging study from the PACS, runs various algorithms, and stores the results (e.g., findings, predictions, and recommendations). (3) A radiologist interprets the study and writes a report. (4) The report goes to the RIS or other clinical system. (5) The information system retrieves the report from the RIS and the imaging study from the PACS, and produces further useful insights and results. For instance, findings may be extracted from the report; various algorithms run on the imaging study; the findings from the report compared with the algorithm results; and discrepancies between the report and algorithm results communicated to clinicians. In a further example, the information system can prospectively notify clinicians of initial results found in Step (2); the information system can also perform retrospective analysis as described in Step (5). (However, in Step (5), the information system can also attempt to retrieve and analyze other prior imaging studies and reports, if available.)

System Applicability in an Example Stroke Scenario.

Consider a scenario where an elderly patient arrives in a primary stroke center with stroke symptoms: such as sudden weakness of right arm; trouble speaking. The patient is not able to provide information on his past medical history. The clinician assesses the symptoms at admission and the neurological assessment (NIHSS score) is provided to the system through the EMR. Symptoms involve limited speech understanding (Vernicke's aphasia).

The patient undergoes a whole brain CT scan with CT perfusion. The acquired data are pushed to the PACS. The broker 116 detects the arrival of the new CT study, fetches it from PACS, and stores it in the patient data cache 112. The patient data cache 112 does not currently contain any other information for this patient, so using the rules engine 114, the broker 116 is called again to fetch patient data from clinical systems 122 (e.g., PACS, EMR, RIS, etc). The broker 116 processes this patient data, extracts relevant details, and stores the results in the patient data cache 112.

The patient data cache 112 now contains the CT Stroke study and more patient data: Symptoms at admission; Neurological assessment; Patient demographic (age, sex, smoking status); and the like. Based on the current contents of the patient data cache 112, the rules engine 114 determines that the following algorithms (from algorithms 118) should be run: an algorithm to search for potential hemorrhage and ischemic lesion (infarct, penumbra). In parallel, the algorithm searches for an occlusion of a major cerebral vessel that could cause the stroke. Using the algorithm, several potential ischemic lesions are automatically detected in the left temporal lobe. The algorithm also finds a suspected occlusion in the M4 MCA territory. The algorithm then stores the locations and measurements in the patient data cache 112.

The results engine 120 produces: (a) visual findings, such as in the form of key images of the suspected ischemic lesions and occluded vessel quantitative findings; (b) volume measurements for putative infarct core and penumbra; (c) initial diagnosis/interpretation (e.g., “acute stroke lesion detected with high probability in Series . . . , no bleeding detected . . . ”); (d) predictive information (e.g., risk of hemorrhagic transformation, predicted recovery score (Modified Rankin Scale—mRS)); and (e) recommendations, such as recommendation for a consultation for suspected cardiac cause of the thrombus.

The clinician can confirm the ischemic lesions and the hemorrhagic stroke exclusion leading to the treatment selection (for example, an intra-arterial thrombolysis). The clinician submits the report to the RIS. The broker 116 detects that a report has become available and fetches it from the RIS data store 122C. The broker 116 processes the report and stores the results in the patient data cache 112. In further examples, feedback and input provided from the clinician to confirm or deny the condition may be used to refine, improve, or modify the algorithm or the algorithm operation in the scenario.

Continuing this example scenario, the patient undergoes an intra-arterial thrombolysis (TPA) and recovers during the following week. The treatment and follow-up results and images are sent to the EMR. The broker 116 detects that a new clinical information for this patient is available and fetches the results, processes them, and stores the results in the patient data cache 112.

Based on the results (thromboembolic stroke) and the other patient data, the rules engine 114 may run other algorithms that produce predictions and treatment recommendations. For example, the rules engine 114 updates the prediction of patient outcome from acute stroke imaging. The rules can also trigger a recommendation for specific cardiac examination and imaging (e.g., Transoesophageal echocardiography) for an evaluation of the left atrial appendage (LAA). The imaging confirms the presence of the existing thrombus as a potential cause of the stroke. A relative risk of recurrent ischemic embolic stroke is computed. The high value (80%) triggers a recommendation to start a new anticoagulant therapy phase in hospital treatment.

System Applicability in an Example Lung Nodule Detection Scenario.

Consider a scenario where a patient undergoes a CT Chest study as part of a screening schedule for lung nodules, which had been discovered in a prior study and are being monitored for growth. The patient is a smoker and has a family history of lung cancer. The broker 116 detects the arrival of the CT Chest study, fetches it from the PACS data store 122A, and stores it in the patient data cache 112. The patient data cache 112 does not currently contain any other information for this patient, so based on the rules engine 114, the broker 116 is called again to fetch patient data from clinical systems (PACS, EMR, RIS, etc). The broker 116 processes this patient data, extracts relevant details, and stores the results in the patient data cache 112.

The patient data cache 112 now contains the CT Chest study and more patient data: gender: female age: 50 smoker: yes medical conditions: high cholesterol prescriptions and medications: statins family history: lung cancer; prior studies: CR study 14 months ago for chest pain; suspected nodule observed in left lung and CT Chest study was recommended. CT Chest study 12 months ago; 2 cm lung nodule was discovered in left lung. Follow up CT Chest study 6 months ago; left lung nodule slightly increased in size compared with last study, new small nodule discovered in right lung.

The Lung CAD algorithm is then run and finds potential lung nodules in the CT Chest study, and matches these nodules with the nodules from the prior studies. The left lung nodule appears to have increased in size to 2.7 cm. The Lung CAD finds the left lung nodule and two nodules in the right lung. Based on the change in the size of the left lung nodule and the medical and family history of the patient, the information system 100 recommends a biopsy.

A radiologist reviews the study, measures the nodules and records their sizes and locations (the left lung nodule is 2.8 cm), and recommends a biopsy. The report goes to the RIS. The broker 116 detects that a report has become available and fetches it from the RIS data store 122C. The broker 116 processes the report and stores the results in the patient data cache 112. Based on this update to the patient data cache 112, the rules engine 114 may run other algorithms, or it may wait for more patient data to become available.

The patient undergoes a biopsy. The biopsy results are entered into the EMR. The broker 116 detects that there is new clinical information for this patient and fetches the biopsy results, processes them, and stores the results in the patient data cache 112. Based on the biopsy results and the rest of the patient data, the rules engine 114 may run other algorithms that produce predictions and treatment recommendations. The rules engine 114 may also request the broker 116 to fetch patient data for patients from similar demographic groups, with similar medical histories, or who have undergone similar procedures to improve its recommendations.

System Applicability in an Example CT Colongraphy Detection Scenario.

Consider a scenario where a patient undergoes a routine CT Colonography study. The patient is a 65-year-old male who has smoked for most of his life and has hypertension and Type 2. Diabetes. He has no history of colon polyps or other lesions, tumors, or nodules.

The CT Colonography study arrives at PACS. The broker 116 detects the arrival of the new CT study, fetches it from the PACS data store 122A, and stores it in the patient data cache 112. The patient data cache 112 does not currently contain any other information for this patient, so based on the rules engine 114, the broker 116 is called again to fetch patient data from clinical systems (PACS, EMR, RIS, etc.). The broker 116 processes this patient data, extracts relevant details, and stores the results in the patient data cache 112.

The patient data cache 112 now contains the CT Colonography study and more patient data: gender: male; age: 65 smoker: yes; medical conditions: type 2 diabetes, hypertension; prescriptions and medications: insulin, blood pressure medications; prior studies: this patient underwent a CT Colonography study 5 years ago. The report noted some vascular calcification, but no colonic polyps or other significant findings.

Based on the current contents of the patient data cache 112, algorithms for landmark detection and contrast detection are run. Both algorithms take the CT Colonography study data from the patient data cache 112 as input. The landmark detection algorithm finds that the abdomen is within the scan extents and stores this result in the patient data cache 112. The contrast detection algorithm finds no contrast within the aorta and stores this result in the patient data cache 112.

Based on these updates to the patient data cache 112, the rules engine 114 determines that the following algorithms should be run: (a) Colon CAD algorithm; (b) aortic aneurysm algorithm for non-contrast CT scans. The Colon CAD algorithm is run on the study and finds potential colon polyps. The locations and measurements from the algorithm are stored the patient data cache 112. The aortic aneurysm algorithm for non-contrast CT scans detects an aortic aneurysm with high probability. (Note, however, that the aortic aneurysm algorithm does not generate measurements.) This algorithm uses the image data from the CT Colonography study along with patient data (male, 65 years old, smoker, type 2 diabetes, hypertension) to form its conclusion. (Note that the aortic aneurysm algorithm could potentially be run on priors to determine if an aortic aneurysm was present at the time of the last study performed on the same patient.)

The results engine 120 produces the following: visual findings: key images of the suspected aortic aneurysm and colon polyps; quantitative findings: measurements and locations of the colon polyps (6 mm polyp in ascending colon, 8 mm polyp in transverse colon, etc.); diagnosis/interpretation: Abdominal aortic aneurysm detected with high probability in Series, colon polyps detected; highest value information: abdominal CT or MR with contrast or US study; recommendations: vascular consultation for suspected abdominal aortic aneurysm

A radiologist reviews the CT Colonography study. The radiologist makes a note of a 6 mm colon polyp in the ascending colon but fails to find any polyps in the transverse colon (hence the 8 mm polyp reported by the Colon CAD was a false positive). The radiologist also observes that there is an aortic aneurysm and measures it. The radiologist records an aortic diameter of 3.9 cm and the location of the AAA and recommends a vascular consultation. The report goes to the RIS.

The broker 116 detects that a new report has become available and fetches it from the RIS data store 122C. The broker 116 processes the report and stores the results in the patient data cache 112. Based on this update to the patient data cache 112, the rules engine 114 may run other algorithms, or it may wait for more patient data to become available. The rules engine 114 may also request that the broker 116 fetch patient data for patients from similar demographic groups, with similar medical histories, or who have undergone similar procedures to improve its recommendations.

Thus, in these scenarios, these and other of the algorithms 118 may be used by the system 110 to detect related pathological conditions in imaging studies even if the primary purpose of the medical diagnostic evaluation was for another condition. These and similar workflows through use of the system 110 can also help reduce missed findings if the algorithms 118 are configured to have high sensitivity. The algorithms 118 may also be adapted to identify clinically relevant findings that users were not looking for and may have otherwise missed, as each of the algorithms 118 may leverage as much available data as possible to produce the most accurate results possible.

Classification Algorithms.

A process for use and development of a classification algorithm may include a sequential process for the analysis of a classifiable medical condition or state. This may include various phases for identification of classifier inputs, definition of classifier operations, and deployment and testing of the classifier. In an example, the identification of inputs for use and development of a classification algorithm may include identifying an abnormality of interest and classification task.

Abnormalities might include characteristics for discrete medical conditions such as aneurysms, fractures, hemorrhages, lesions, nodules, tumors, polyps, and so forth. Classification tasks include, but are not limited to, aspects of: detection (e.g., does the study exhibit the abnormality); localization (e.g., approximately where is the abnormality, at a location in the image, the slice number in an imaging series, etc.); segmentation and quantification (e.g., automatically segment the abnormality and compute quantitative measurements such as size, diameter, density, or volume); characterization (e.g., what is the stage, grade, type, malignancy, of the condition); prognosis (e.g., what is the predicted patient outcome?); and recommendations (e.g., based on data values for the classification tasks, what clinical care is defined or associated with treatment options?).

The identification of inputs for use and development of a classification algorithm may include the identification of applicable types and formats of patient data within the system 110. As discussed above, patient data includes, but is not limited to, imaging studies, EMR/RIS/HIS data, laboratory test results, pathology reports, genomics, and so forth. Certain types of and values from the patient data may be associated with a definition for the classification algorithm, to develop rules for accurately identifying such patient data, and identify other algorithms that are needed to accurately identify applicable patient data. Further, such inputs may be used to prevent the execution of classification algorithms when such algorithms are not relevant (thus, preventing waste of system resources and reducing the risk of false alarms). In still further examples, considerations such as medical facility or physician preferences, regulatory constraints, or the like may be used as rules to prevent or increase the execution of one or more classification algorithms.

Further definition for the classification algorithm may include the collection of patient data examples to use for training and validation, having sufficiently large numbers of positive (abnormal) and negative (healthy) examples (e.g., from a developed training data set). From this training data, the one or more classification algorithms may be developed to perform the classification task, the algorithm(s) may have dependencies on one or more other algorithms, in which case there is a pipeline of algorithms whose combined performance must be rigorously evaluated. Configurable parameters may also be defined, as such parameters are considered during evaluation of the performance of the algorithm. Finally, a total time needed to execute the algorithm should be acceptable given the type of task that it performs and the types of studies that the algorithm operates on.

In an example, the deployment and testing of the classified algorithm may include specialized programming used to integrate features of a classification or analysis algorithm, and algorithm results, into a target deployment environment or user interface. In a further example, the target deployment environment may include an advanced image visualization system, an image modality, an image data management system (e.g., a PACS), a clinical workstation system, or a standalone computing system. Use of the results and insights may also extend to any number or forms of systems that are networked and adapted to receive data, such as to automatically execute algorithms when new patient data becomes available, and to automatically use of algorithm results (communicating results to users, using results to run other algorithms, and so forth).

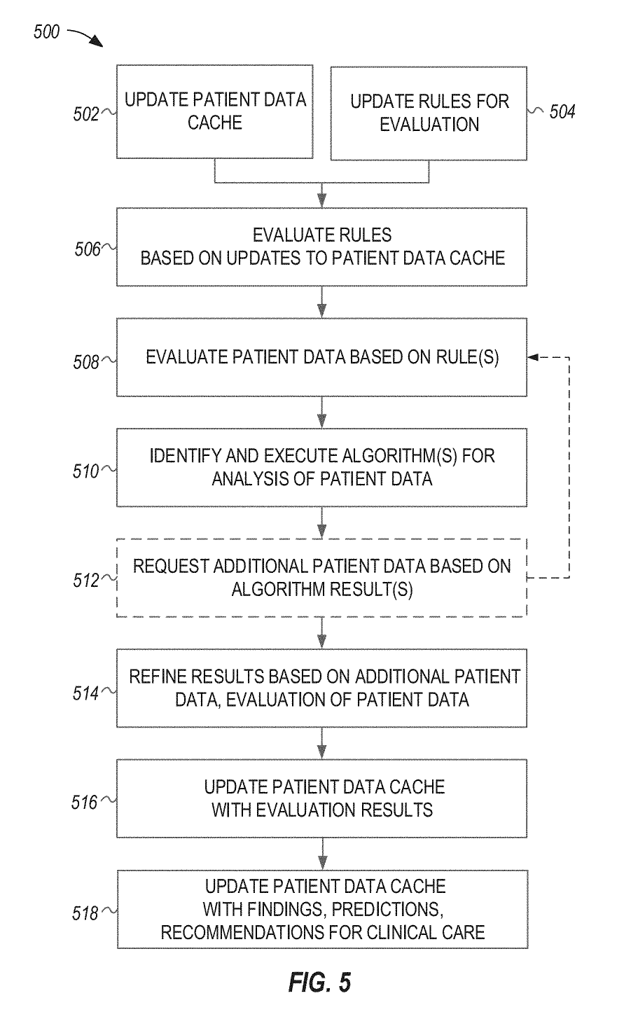

FIG. 5 illustrates a flowchart 500 of example sequential data processing operations performed for obtaining and analyzing data in a clinical data analysis system. As shown, the flowchart 500 depicts a set of sequential operations, which start from the initial collection of data and rules for operation of an automated insight information system. It will be understood that the sequence of activities portrayed in FIG. 5 is provided for purposes of illustration, and many more detailed activities (including activities provided in another order or sequence, and the interrelationship between different data-derived actions) may occur with the operations at one or multiple system locations.

The flowchart 500 initially depicts the update of a patient data cache (operation 502) and the update rules for evaluation (operation 504). In an initial state, these operations may include providing or obtaining the patient data and the rules from an initial state. Based on the provided or updated information, a rules engine may proceed to identify and evaluate the rules applicable to a particular data set, based on the provision or updates to the data set in the patient data cache (operation 506).

Based on the evaluated rules, various forms and types of data may be identified and selected for evaluation from the patient data cache (operation 508). One or more algorithms for analysis of the patient data, which are capable of analyzing the identified and selected data according to the evaluated rules, are then identified and executed (operation 510). The evaluation of rules, patient data, and execution of the algorithms may include implementation of the operations with a broker, rules engine, patient data cache, and results engine, discussed above with reference to FIGS. 3 and 4, and the system coordination operations discussed above with reference to FIGS. 1 to 2B.

Based on the results of the algorithm, additional (new, supplemental) patient data may be requested (operation 512, indicated as optional). The obtaining of new patient data may lead to the repeat evaluation of the new patient data (operation 508) and the identification and execution of additional algorithms to evaluate the new patient data (operation 510).

The flowchart 500 further depicts operations that will refine results (e.g., prepare, update, or classify findings, recommendations, predictions) based on any additional patient data, and based on the patient data, and any rules or conditions for post-processing (operation 514). This is followed by the storage or update of the algorithm results (evaluation results) to the patient data cache (operation 516), and the storage or update of the patient data cache with specific findings, predictions, and recommendations for clinical care (operation 518). Additional processing may be utilized to convert the algorithm results and findings (and multiple of such results in findings) into specific predictions and recommendations relating to one or more medical conditions.

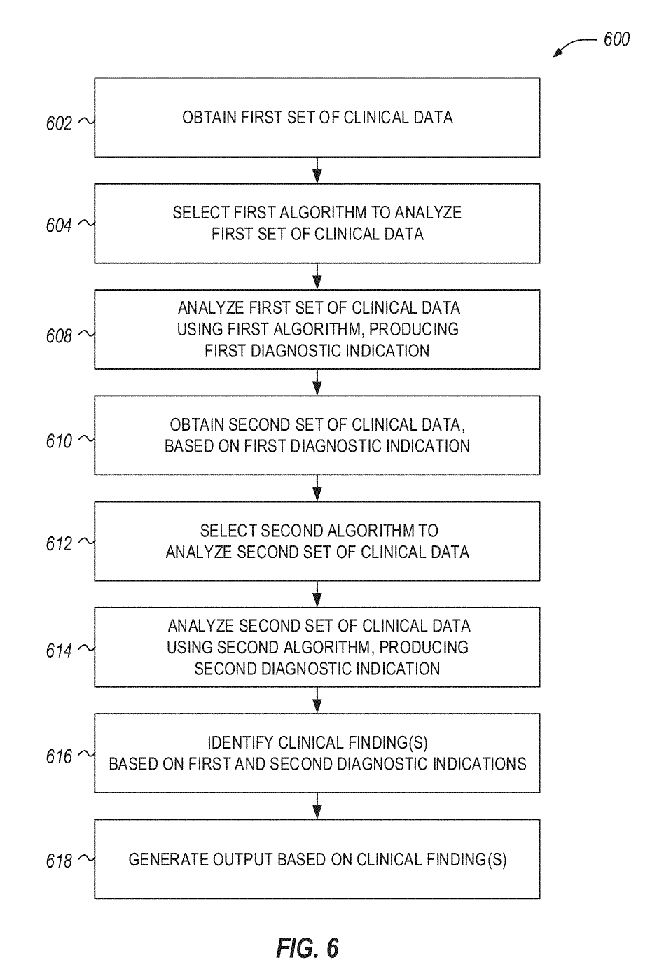

FIG. 6 illustrates a flowchart of an example method performed by a computing system for information processing in a computing system, such as performed by an automated insight information system. This flowchart 600 provides a high-level depiction of operations used to obtain, process, and output data in such a system, but it will be understood that additional operations (including the integration of the operations from flowchart 500 and operations of the system 110 as illustrated in FIGS. 1 to 4) may be implemented into the depicted flow.

In an example, the operations depicted in the flowchart 600 include the obtaining of a first set of clinical data (operation 602), such as clinical data that is provided (e.g., requested and received) from a first data source. Based on the type of clinical data, and medical (e.g., diagnostic) properties that are provided or directly indicated in the first set of clinical data, a first algorithm is selected to analyze the first set of clinical data (operation 604). This first algorithm is utilized to analyze the first set of clinical data, and the first algorithm then produces a first diagnostic indication from characterizing at least part of the first set of clinical data. In a further example, characterizing includes applying a classification, a rule set, or other evaluative condition on the clinical data.

As further shown in the flowchart 600, subsequent operations proceed for a second set of clinical data. This may include obtaining (e.g., requesting and receiving) of a second set of clinical data (operation 610), which may be selected or requested on the basis at least one clinical property provided in the first set of clinical data, or information provided in the first diagnostic indication. In specific examples, the first electronic data source is a first type of a clinical data source, whereas the second electronic data source is a different second type of a clinical data source, provided from among: a picture archiving communications system (PACS), electronic medical record (EMR) system, a laboratory information system, a radiology information system (RIS), a pathology information system, or a vendor neutral archive (VNA). In another example, the first and second electronic data sources are the same type of systems hosted by the same (or different) locations. In a further example, the second data source may be selected based on the first diagnostic indication produced from the first algorithm.

Based on the type of the second clinical data, and medical (e.g., diagnostic) properties provided or directly indicated in the second set of clinical data, a second algorithm is selected to analyze the second set of clinical data (operation 612). This second algorithm is utilized to analyze the second set of clinical data, and the second algorithm then produces a second diagnostic indication based on characterizing (e.g., classifying, applying rules, etc.) at least part of the second set of clinical data (operation 614). In a specific example, the first algorithm and the second algorithm are respective machine learning models, and the first algorithm is trained to evaluate a different set of anatomical features and a different diagnostic medical condition than the second algorithm.

Based on the first and second diagnostic indications, one or more clinical findings may be produced and identified (operation 616). Output from the information system then may be provided based on the one or more clinical findings (operation 618). In specific examples, a graphical output of an identified clinical finding may be produced, with the graphical output including diagnostic information relating to at least one of: visual findings, quantitative findings, diagnosis of a medical condition, an indication of highest value information from the first or second set of clinical data, predictions of the medical condition, recommended tests of the medical condition, or recommended treatments of the medical condition.

In further examples, operations may be performed that select one or more subsequent analytical algorithms based on results from the second algorithm, including repeating operations for requesting a subsequent set of clinical data from a subsequent electronic data source, and analyzing the subsequent set of clinical data using the subsequent analytical algorithms. For instance, the one or more subsequent analytical algorithms may produce a subsequent set of algorithm results, and the clinical finding (e.g., produced in operations 616, 618) may be further based on the subsequent set of algorithm results.

Also in further examples, the first set and second sets of clinical data may be transformed into a standardized and structured format (or, different formats from each other). Also in further examples, a patient data cache may be used to store the clinical data sets, the clinical information used to select the first and the second algorithm, the clinical properties or diagnostic indications identified by the algorithm, or the clinical findings. Also in further examples, the first algorithm and the second algorithm produce respective sets of processing results, that are stored the a patient data cache, and operations for identifying the clinical finding are performed based on a combination of the respective sets of processing results. Also in further examples, a number of additional algorithms (or algorithm sub-processes) may be invoked to allow the first algorithm to provide the first diagnostic indication based on data results from the additional algorithms, such that the characterizing of the clinical data is performed based on the data results produced from executing the additional algorithms/sub-processes on respective portions of the clinical data.

FIG. 7 illustrates a block diagram of components in a system 700 used for information processing, for an example use case of automated insight information processing. For example, the system 700 may include: an information processing computing system 702 (e.g., a server or client system) configured to continuously monitor clinical systems for new patient data, process patient data into a standardized and structured format, selectively run algorithms to classify and characterize data, and store the results of algorithms using the techniques described herein; a data source computing system 730 (e.g., a server or other a centralized system) configured to provide the patient data using the techniques described herein; and a data output computing system 740 configured to present or provide information from the algorithm analysis to other systems and users using the techniques described herein. The following examples specifically encompass configuration of the information processing computing system 702 and the data source computing system 730 to interact and iteratively request additional data as the information processing computing system 702 operates additional algorithms and produces additional insight results; however, it will be understood that the system configuration discussed herein is applicable to other types of workflows, data processing, data storage, and algorithm deployments in medical and non-medical deployments.

The information processing computing system 702 may include components (e.g., programmed or specially arranged circuitry) for implementing processing functionality with: an algorithm data library 704 that hosts and provides the algorithms discussed herein; a rules engine 706 that defines rules and like conditions and prerequisites for operation of the algorithm(s) as discussed herein; a data request engine 708 that requests and obtains data from the clinical data sources as discussed herein; a results engine 710 that coordinates the collection of results produced from the execution of respective algorithm(s) as discussed herein; a data cache 712 that stores and persists data and results within the system 702; and a rules library 714 that provides a set of rules (e.g., prerequisites, conditions) for the evaluation and application of particular algorithms.

The information processing computing system 702 may further include an information output component 716 to provide output of results (and coordinated findings, recommendations, and predictions) generated from the information system in one or more formats. The information output component 716 may specifically implement functionality such as: findings output functionality 718 (e.g., for identifying specific findings related to medical conditions, diagnostic statuses, human anatomical features, structures, or characteristics); prediction output functionality 720 (e.g., for quantifying or indicating a predictive state of a particular medical condition or diagnostic status); recommendation output processing 722 (e.g., for generating recommendations and instructions related to); and workflow modification functionality 724 (e.g., for implementing changes in diagnostic or clinical processing workflows, processes, and related actions based on the results).

The information processing computing system 702 may further include electronic components for user input, output, and processing, such as processing circuitry 728, a graphical user interface 726, an output device 729A (e.g., to provide output of the graphical user interface); and an input device 729B (e.g., to provide input for workflow or control activities in the graphical user interface 726). In a further example, the graphical user interface 726, the output device 729A, and the input device 729B are used to establish, engage, or modify features of the algorithms, rules, or rules, to further interact with features of the algorithm-based information processing workflow using the techniques described herein.

The data source computing system 730 may include data request processing 732 and one or more databases 734. In an example, the data request processing 732 is adapted to receive, access, and provide clinical data, in response to data requests, and the one or more databases 734 are adapted to host the requested data and like forms of clinical data. In other examples (not depicted), additional databases or data source computing systems may be utilized, such as in cases where separate types of medical data is stored separately. The data source computing system 730 may also be responsible for obtaining, hosting, or generating the data as such data is created in the appropriate medical setting.

The data output computing system 740 may include functionality for image visualization processing 744 and result visualization processing 746, which may integrate with use of a graphical user interface 742, such as for generation of a dashboard or other graphical output of identified clinical findings. The output of such a graphical user interface 742 and the processing components 744, 746 may be provided through use of processing circuitry 748, input device 749B, and output device 749A. In another example, the features of the data output computing system 740 may be integrated or combined with the features of the information processing computing system 702 or other centralized processing components.

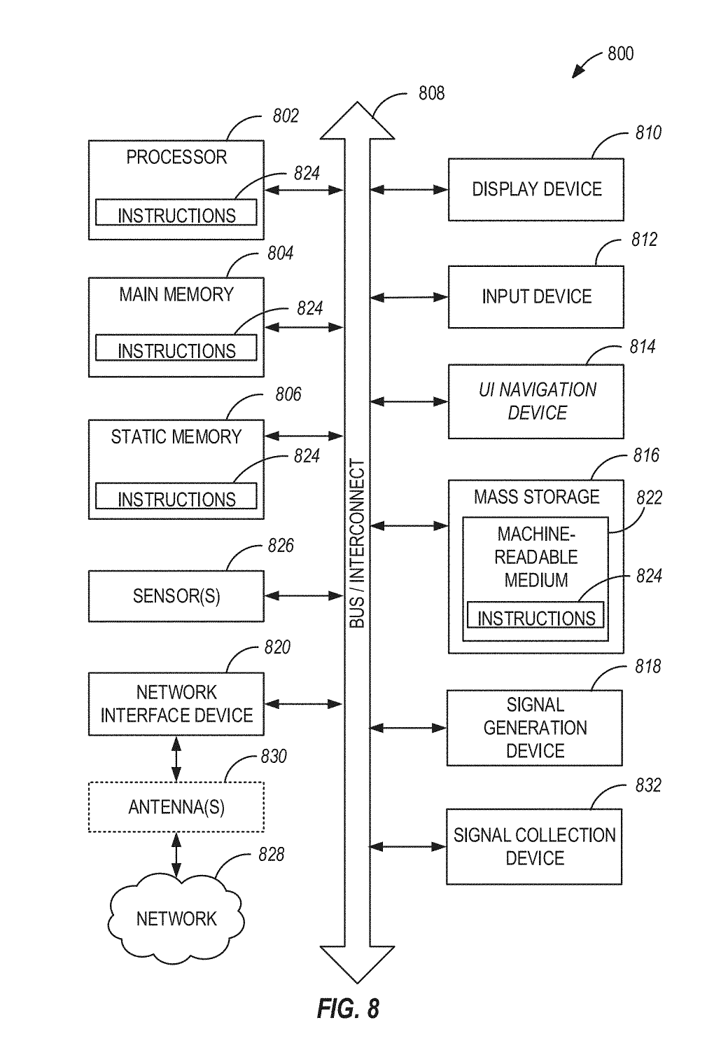

FIG. 8 is a block diagram illustrating an example computing system machine upon which any one or more of the methodologies herein discussed may be run. Computer system 800 may be embodied as a computing device, providing operations of the components featured in the various figures, including components of the automated insight information system 110, the data sources 122, the dashboard 130, the information processing computing system 702, the data source computing system 730, the data output computing system 740, or as an execution platform for the operations in flowcharts 500 and 600, or any other processing, storage, or computing platform or component described or referred to herein. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The computer system machine may be a personal computer (PC) that may or may not be portable (e.g., a notebook or a netbook), a tablet, a Personal Digital Assistant (PDA), a mobile telephone or smartphone, a thin client, a web appliance, a virtual machine host, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

Example computer system 800 includes a processor 802 (e.g., a central processing unit (CPU), a graphics processing unit (GPU) or both), a main memory 804 and a static memory 806, which communicate with each other via an interconnect 808 (e.g., a link, a bus, etc.). The computer system 800 may further include a video display unit 810, an alphanumeric input device 812 (e.g., a keyboard), and a user interface (UI) navigation device 814 (e.g., a mouse). In one example, the video display unit 810, input device 812 and UI navigation device 814 are a touch screen display. The computer system 800 may additionally include a storage device 816 (e.g., a drive unit), a signal generation device 818 (e.g., a speaker), a signal collection device 832, and a network interface device 820 (which may include or operably communicate with one or more antennas 830, transceivers, or other wireless communications hardware), and one or more sensors 826.

The storage device 816 includes a machine-readable medium 822 on which is stored one or more sets of data structures and instructions 824 (e.g., software) embodying or utilized by any one or more of the methodologies or functions described herein. The instructions 824 may also reside, completely or at least partially, within the main memory 804, static memory 806, and/or within the processor 802 during execution thereof by the computer system 800, with the main memory 804, static memory 806, and the processor 802 also constituting machine-readable media.

While the machine-readable medium 822 is illustrated in an example to be a single medium, the term “machine-readable medium” may include a single medium or multiple media (e.g., a centralized or distributed database, and/or associated caches and servers) that store the one or more instructions 824. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, optical media, and magnetic media. Specific examples of machine-readable media include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 824 may further be transmitted or received over a communications network 828 using a transmission medium via the network interface device 820 utilizing any one of a number of well-known transfer protocols (e.g., HTTP). Examples of communication networks include a local area network (LAN), wide area network (WAN), the Internet, mobile telephone networks, Plain Old Telephone (POTS) networks, and wireless data networks (e.g., Wi-Fi, 3G, and 4G LTE/LTE-A or WiMAX networks). Such communications may also be facilitated using any number of personal area networks, LANs, and WANs, using any combination of wired or wireless transmission mediums. The term “transmission medium” shall be taken to include any intangible medium that is capable of storing, encoding, or carrying instructions for execution by the machine, and includes digital or analog communications signals or other intangible medium to facilitate communication of such software.

The embodiments described above may be implemented in one or a combination of hardware, firmware, and software. While some embodiments described herein illustrate only a single machine or device, the terms “system”, “machine”, or “device” shall also be taken to include any collection of machines or devices that individually or jointly execute a set (or multiple sets) of instructions to perform any one or more of the methodologies discussed herein.

Examples, as described herein, may include, or may operate on, logic or a number of components, modules, or mechanisms. Such components may be tangible entities (e.g., hardware) capable of performing specified operations and may be configured or arranged in a certain manner. In an example, circuits may be arranged (e.g., internally or with respect to external entities such as other circuits) in a specified manner to implement such components. In an example, the whole or part of one or more computer systems (e.g., a standalone, client or server computer system) or one or more hardware processors may be configured by firmware or software (e.g., instructions, an application portion, or an application) that operates to perform specified operations. In an example, the software may reside on a machine readable medium. In an example, the software, when executed by the underlying hardware, causes the hardware to perform the specified operations.

Accordingly, such components may be a tangible entity that is physically constructed, specifically configured (e.g., hardwired), or temporarily (e.g., transitorily) configured (e.g., programmed) to operate in a specified manner or to perform part or all of any operation described herein. Considering examples in which such components are temporarily configured, each of the components need not be instantiated at any one moment in time. For example, where the components comprise a general-purpose hardware processor configured using software, the general-purpose hardware processor may be configured as respective different components at different times. Software may accordingly configure a hardware processor, for example, to constitute a particular component at one instance of time and to constitute a different component at a different instance of time.

Additional examples of the presently described method, system, and device embodiments are suggested according to the structures and techniques described above and specified in the following claims.

For example, Example 1 of the subject matter described herein may be embodied by a method for information processing in a computing system, performed by electronic operations executed by processing circuitry of the computing system, with the electronic operations comprising: obtaining a first set of clinical data associated with a patient from a first electronic data source; selecting a first algorithm to analyze the first set of clinical data, the first algorithm selected from a library of algorithms based on at least one clinical property provided in the first set of clinical data; analyzing the first set of clinical data using the first algorithm, wherein the first algorithm produces a first diagnostic indication based on characterizing at least part of the first set of clinical data; obtaining a second set of clinical data associated with the patient from a second electronic data source; selecting a second algorithm to analyze the second set of clinical data, the second algorithm selected from the library of algorithms based on at least one clinical property provided in the first set of clinical data; analyzing the second set of clinical data using the second algorithm, wherein the second algorithm produces a second diagnostic indication based on characterizing at least part of the second set of clinical data; identifying a clinical finding based on the first diagnostic indication and the second diagnostic indication; and generating output from the computing system based on the identified clinical finding.

In Example 2, the subject matter of Example 1 includes, wherein the first electronic data source is a first type, and the second electronic data source is a different second type, of: a picture archiving communications system (PACS), electronic medical record (EMR) system, a laboratory information system, a radiology information system (RIS), a pathology information system, or a vendor neutral archive (VNA); wherein the second data source is selected based on the first diagnostic indication; and wherein obtaining the first and second sets of clinical data includes requesting and receiving the first and second sets of clinical data from the respective first and second electronic data sources.

In Example 3, the subject matter of Examples 1-2 includes, wherein the second algorithm is further selected from the library of algorithms based on at least one clinical property produced from the first algorithm; wherein the first algorithm and the second algorithm are respective machine learning models, and wherein characterizing the first and second sets of clinical data includes classifying the first and second sets of data with the respective first and second algorithms; and wherein the first algorithm is trained to evaluate a different set of anatomical features and a different diagnostic medical condition than the second algorithm.

In Example 4, the subject matter of Example 3 includes, the electronic operations comprising: selecting one or more subsequent analytical algorithms based on results from the second algorithm; obtaining a subsequent set of clinical data from a subsequent electronic data source; and analyzing the subsequent set of clinical data using the subsequent analytical algorithms, wherein the one or more subsequent analytical algorithms produce a subsequent set of algorithm results; wherein the clinical finding is further based on the subsequent set of algorithm results.

In Example 5, the subject matter of Examples 1-4 includes, the electronic operations further comprising: in response to obtaining the first set of clinical data, transforming the first set of clinical data into a standardized and structured format; and in response to obtaining the second set of clinical data, transforming the second set of clinical data into the standardized and structured format; wherein an original format of the first set of clinical data and the original format of the second set of clinical data differ from each other and from the standardized and structured format.

In Example 6, the subject matter of Examples 1-5 includes, the electronic operations further comprising: storing, in a patient data cache, the first set of clinical data and the second set of clinical data, storing, in the patient data cache, the at least one clinical property provided in the first set of clinical data that is used to select the first algorithm and the second algorithm; storing, in the patient data cache, the first diagnostic indication and the second diagnostic indication; and storing, in the patient data cache, the clinical finding based on the first diagnostic indication and the second diagnostic indication.

In Example 7, the subject matter of Examples 1-6 includes, wherein the first algorithm and the second algorithm further produce respective sets of processing results, to be stored in a patient data cache, and wherein identifying the clinical finding is based on a combination of the respective sets of processing results; and wherein the output from the computing system based on the identified clinical findings includes one or more clinical predictions or clinical recommendations produced from the clinical finding.

In Example 8, the subject matter of Examples 1-7 includes, wherein the first set of clinical data includes medical imaging data that represents one or more human anatomical features in one or more medical images; wherein the second set of clinical data includes textual data that represents medical information relating to a medical condition for the one or more human anatomical features depicted in the one or more medical images; wherein the first algorithm performs detection, segmentation, quantification, or prediction operations on the one or more medical images; and wherein the second algorithm performs a diagnostic evaluation on characteristics of the textual data, in response to the detection, segmentation, quantification, or prediction operations on the one or more medical images.

In Example 9, the subject matter of Examples 1-8 includes, wherein the second algorithm is selected based on a defined set of prerequisites, wherein the defined set of prerequisites includes a first prerequisite that is satisfied at least in part by the first diagnostic indication.

In Example 10, the subject matter of Examples 1-9 includes, wherein the first algorithm produces the first diagnostic indication based on data results produced from a plurality of additional algorithms, wherein the characterizing of the at least part of the first set of clinical data is performed based on the data results produced from executing the plurality of additional algorithms on respective portions of the first set of clinical data.

In Example 11, the subject matter of Examples 1-10 includes, wherein generating the output from the computing system includes: generating a graphical output of an identified clinical finding, the graphical output including diagnostic information relating to at least one of: visual findings, quantitative findings, diagnosis of a medical condition, an indication of highest value information from the first or second set of clinical data, predictions of the medical condition, recommended tests of the medical condition, or recommended treatments of the medical condition.

In Example 12, the subject matter of Examples 1-11 includes, the electronic operations further comprising: modifying a medical information workflow, in response to the clinical finding, wherein the medical information workflow is a diagnostic workflow used to evaluate at least the first set of clinical data.

Example 13 is at least one machine readable medium including instructions, which when executed by a computing system, cause the computing system to perform any of the methods of Examples 1-12.

Example 14 is an apparatus comprising means for performing any of the methods of Examples 1-12.

Example 15 is a computing system, comprising storage device to store a set of instructions, and processing circuitry including at least one processor to execute the set of instructions, wherein the set of instructions are provided from a plurality of components, and wherein the processing circuitry is arranged to execute instructions with the at least one processor to execute the instructions from the plurality of components to perform any of the methods of Examples 1-12.

Other non-limiting examples may be configured to operate separately, or can be combined in any permutation or combination with any one or more of the other examples provided above, in the following claims, or throughout the present disclosure.

What is claimed is:

1. A method for information processing in a computing system, performed by electronic operations executed by processing circuitry of the computing system, with the electronic operations comprising:
obtaining a first set of clinical data associated with a patient from a first electronic data source;
selecting a first algorithm to analyze the first set of clinical data, the first algorithm selected from a library of algorithms based on at least one clinical property provided in the first set of clinical data;
analyzing the first set of clinical data using the first algorithm, wherein the first algorithm produces a first diagnostic indication based on characterizing at least part of the first set of clinical data;
obtaining a second set of clinical data associated with the patient from a second electronic data source;
selecting a second algorithm to analyze the second set of clinical data, the second algorithm selected from the library of algorithms based on at least one clinical property provided in the first set of clinical data;
analyzing the second set of clinical data using the second algorithm, wherein the second algorithm produces a second diagnostic indication based on characterizing at least part of the second set of clinical data;
identifying a clinical finding based on the first diagnostic indication and the second diagnostic indication; and
generating output from the computing system based on the identified clinical finding.

2. The method of claim 1,
wherein the first electronic data source is a first type, and the second electronic data source is a different second type, of: a picture archiving communications system (PACS), electronic medical record (EMR) system, a laboratory information system, a radiology information system (RIS), a pathology information system, or a vendor neutral archive (VNA);
wherein the second data source is selected based on the first diagnostic indication; and
wherein obtaining the first and second sets of clinical data includes requesting and receiving the first and second sets of clinical data from the respective first and second electronic data sources.

3. The method of claim 1,
wherein the second algorithm is further selected from the library of algorithms based on at least one clinical property produced from the first algorithm;
wherein the first algorithm and the second algorithm are respective machine learning models, and wherein characterizing the first and second sets of clinical data includes classifying the first and second sets of data with the respective first and second algorithms; and
wherein the first algorithm is trained to evaluate a different set of anatomical features and a different diagnostic medical condition than the second algorithm.

4. The method of claim 3, the electronic operations comprising:
selecting one or more subsequent analytical algorithms based on results from the second algorithm;
obtaining a subsequent set of clinical data from a subsequent electronic data source; and
analyzing the subsequent set of clinical data using the subsequent analytical algorithms, wherein the one or more subsequent analytical algorithms produce a subsequent set of algorithm results;
wherein the clinical finding is further based on the subsequent set of algorithm results.

5. The method of claim 1, the electronic operations further comprising:
in response to obtaining the first set of clinical data, transforming the first set of clinical data into a standardized and structured format; and
in response to obtaining the second set of clinical data, transforming the second set of clinical data into the standardized and structured format;
wherein an original format of the first set of clinical data and the original format of the second set of clinical data differ from each other and from the standardized and structured format.

6. The method of claim 1, the electronic operations further comprising:
storing, in a patient data cache, the first set of clinical data and the second set of clinical data;
storing, in the patient data cache, the at least one clinical property provided in the first set of clinical data that is used to select the first algorithm and the second algorithm;
storing, in the patient data cache, the first diagnostic indication and the second diagnostic indication; and
storing, in the patient data cache, the clinical finding based on the first diagnostic indication and the second diagnostic indication.

7. The method of claim 1,
wherein the first algorithm and the second algorithm further produce respective sets of processing results, to be stored in a patient data cache, and wherein identifying the clinical finding is based on a combination of the respective sets of processing results; and
wherein the output from the computing system based on the identified clinical findings includes one or more clinical predictions or clinical recommendations produced from the clinical finding.

8. The method of claim 1,
wherein the first set of clinical data includes medical imaging data that represents one or more human anatomical features in one or more medical images;
wherein the second set of clinical data includes textual data that represents medical information relating to a medical condition for the one or more human anatomical features depicted in the one or more medical images;
wherein the first algorithm performs detection, segmentation, quantification, or prediction operations on the one or more medical images; and
wherein the second algorithm performs a diagnostic evaluation on characteristics of the textual data, in response to the detection, segmentation, quantification, or prediction operations on the one or more medical images.

9. The method of claim 1,
wherein the second algorithm is selected based on a defined set of prerequisites, wherein the defined set of prerequisites includes a first prerequisite that is satisfied at least in part by the first diagnostic indication.

10. The method of claim 1,
wherein the first algorithm produces the first diagnostic indication based on data results produced from a plurality of additional algorithms, wherein the characterizing of the at least part of the first set of clinical data is performed based on the data results produced from executing the plurality of additional algorithms on respective portions of the first set of clinical data.

11. The method of claim 1, wherein generating the output from the computing system includes:
generating a graphical output of an identified clinical finding, the graphical output including diagnostic information relating to at least one of: visual findings, quantitative findings, diagnosis of a medical condition, an indication of highest value information from the first or second set of clinical data, predictions of the medical condition, recommended tests of the medical condition, or recommended treatments of the medical condition.

12. The method of claim 1, the electronic operations further comprising:
modifying a medical information workflow, in response to the clinical finding, wherein the medical information workflow is a diagnostic workflow used to evaluate at least the first set of clinical data.

13. At least non-transitory machine-readable medium, the machine-readable medium including instructions, which when executed by a computing system having a hardware processor, causes the processor to perform operations for clinical information processing, the operations comprising:
obtaining a first set of clinical data associated with a patient from a first electronic data source;
selecting a first algorithm to analyze the first set of clinical data, the first algorithm selected from a library of algorithms based on at least one clinical property provided in the first set of clinical data;
analyzing the first set of clinical data using the first algorithm, wherein the first algorithm produces a first diagnostic indication based on characterizing the first set of clinical data;
obtaining a second set of clinical data associated with the patient from a second electronic data source;
selecting a second algorithm to analyze the second set of clinical data, the second algorithm selected from the library of algorithms based on at least one clinical property provided in the first set of clinical data;
analyzing the second set of clinical data using the second algorithm, wherein the second algorithm produces a second diagnostic indication based on characterizing the second set of clinical data;
identifying a clinical finding based on the first diagnostic indication and the second diagnostic indication; and
generating output from the computing system based on the identified clinical finding.

14. The machine-readable medium of claim 13,
wherein the first electronic data source is a first type, and the second electronic data source is a different second type, of: a picture archiving communications system (PACS), electronic medical record (EMR) system, a laboratory information system, a radiology information system (RIS), a pathology information system, or a vendor neutral archive (VNA);
wherein the second data source is selected based on the first diagnostic indication; and
wherein obtaining the first and second sets of clinical data includes requesting and receiving the first and second sets of clinical data from the respective first and second electronic data sources.

15. The machine-readable medium of claim 13,
wherein the second algorithm is further selected from the library of algorithms based on at least one clinical property produced from the first algorithm;
wherein the first algorithm and the second algorithm are respective machine learning models, and wherein characterizing the first and second sets of clinical data includes classifying the first and second sets of data with the respective first and second algorithms; and
wherein the first algorithm is trained to evaluate a different set of anatomical features and a different diagnostic medical condition than the second algorithm.

16. The machine-readable medium of claim 15, the operations further comprising:
selecting one or more subsequent analytical algorithms based on results from the second algorithm;
obtaining a subsequent set of clinical data from a subsequent electronic data source; and
analyzing the subsequent set of clinical data using the subsequent analytical algorithms, wherein the one or more subsequent analytical algorithms produce a subsequent set of algorithm results;
wherein the clinical finding is further based on the subsequent set of algorithm results.

17. The machine-readable medium of claim 13, the operations further comprising:
in response to obtaining the first set of clinical data, transforming the first set of clinical data into a standardized and structured format; and
in response to obtaining the second set of clinical data, transforming the second set of clinical data into the standardized and structured format;
wherein an original format of the first set of clinical data and the original format of the second set of clinical data differ from each other and from the standardized and structured format.

18. The machine-readable medium of claim 13, the operations further comprising:
storing, in a patient data cache, the first set of clinical data and the second set of clinical data; storing, in the patient data cache, the at least one clinical property provided in the first set of clinical data that is used to select the first algorithm and the second algorithm;
storing, in the patient data cache, the first diagnostic indication and the second diagnostic indication; and
storing, in the patient data cache, the clinical finding based on the first diagnostic indication and the second diagnostic indication.

19. The machine-readable medium of claim 13,
wherein the first algorithm and the second algorithm further produce respective sets of processing results, to be stored in a patient data cache, and wherein identifying the clinical finding is based on a combination of the respective sets of processing results; and
wherein the output from the computing system based on the identified clinical findings includes one or more clinical predictions or clinical recommendations produced from the clinical finding.

20. The machine-readable medium of claim 13, the medium including instructions that cause the machine to perform operations that:
wherein the first set of clinical data includes medical imaging data that represents one or more human anatomical features in one or more medical images;
wherein the second set of clinical data includes textual data that represents medical information relating to a medical condition for the one or more human anatomical features depicted in the one or more medical images;
wherein the first algorithm performs detection, segmentation, quantification, or prediction operations on the one or more medical images; and
wherein the second algorithm performs a diagnostic evaluation on characteristics of the textual data, in response to the detection, segmentation, quantification, or prediction operations on the one or more medical images.

21. The machine-readable medium of claim 13,
wherein the second algorithm is selected based on a defined set of prerequisites, wherein the defined set of prerequisites includes a first prerequisite that is satisfied at least in part by the first diagnostic indication.

22. The machine-readable medium of claim 13,
wherein the first algorithm produces the first diagnostic indication based on data results produced from a plurality of additional algorithms, wherein the characterizing of the at least part of the first set of clinical data is performed based on the data results produced from executing the plurality of additional algorithms on respective portions of the first set of clinical data.

23. The machine-readable medium of claim 13, wherein generating the output from the computing system includes:
generating a graphical output of identified clinical finding, the graphical output including diagnostic information relating to at least one of: visual findings, quantitative findings, diagnosis of a medical condition, an indication of highest value information from the first or second set of clinical data, predictions of the medical condition, recommended tests of the medical condition, or recommended treatments of the medical condition.

24. The machine-readable medium of claim 13, the operations further comprising:
modifying a medical information workflow, in response to the clinical finding, wherein the medical information workflow is a diagnostic workflow used to evaluate at least the first set of clinical data.

25. An information processing system, comprising:
a storage device to store a set of instructions; and
processing circuitry including at least one processor to execute the set of instructions, wherein the set of instructions are provided from a plurality of components including:
a data request engine, the data request engine operable to:
request and receive a first set of clinical data associated with a patient from a first electronic data source; and
request and receive a second set of clinical data associated with the patient from a second electronic data source;
an algorithm data library, the algorithm data library including a plurality of executable algorithms, wherein the executable algorithms are obtained from the algorithm data library to:
identify a first algorithm to analyze the first set of clinical data, the first algorithm identified from a library of algorithms based on at least one clinical property provided in the first set of clinical data;
analyze the first set of clinical data using the first algorithm, wherein the first algorithm produces a first diagnostic indication based on characterizing at least part of the first set of clinical data;
identify a second algorithm to analyze the second set of clinical data, the second algorithm identified from the library of algorithms based on at least one clinical property provided in the first set of clinical data; and
analyze the second set of clinical data using the second algorithm, wherein the second algorithm produces a second diagnostic indication based on characterizing at least part of the second set of clinical data;
a results engine, the results engine operable to:
identify a clinical finding based on the first diagnostic indication and the second diagnostic indication; and
generate output from the information processing system based on the identified clinical finding.

26. The system of claim 25,
wherein the algorithm data library is further operable to select one or more subsequent analytical algorithms based on results from the second algorithm;
wherein the data request engine is further operable to request a subsequent set of clinical data from a subsequent electronic data source;
wherein the algorithm data library is further operable to analyze the subsequent set of clinical data using the subsequent analytical algorithms, wherein the one or more subsequent analytical algorithms produce a subsequent set of algorithm results;
wherein the clinical finding is further based on the subsequent set of algorithm results; and
wherein the first electronic data source is a first type, and the second electronic data source is a different second type, of: a picture archiving communications system (PACS), electronic medical record (EMR) system, a laboratory information system, a radiology information system (RIS), a pathology information system, or a vendor neutral archive (VNA).

27. The system of claim 25, the plurality of components further including:
an information output component operable to generate a graphical output of identified clinical finding, the graphical output including diagnostic information relating to at least one of: visual findings, quantitative findings, diagnosis of a medical condition, an indication of highest value information from the first or second set of clinical data, predictions of the medical condition, recommended tests of the medical condition, or recommended treatments of the medical condition;
wherein identifying the clinical finding is based on a combination of the respective sets of processing results; and
wherein the output from the information processing system based on the identified clinical findings includes one or more clinical predictions or clinical recommendations produced from the clinical finding.
